(19) United States
(12) Patent Application Publication
(10) Pub. No.: US 2012/0142023 A1
(43) Pub. Date: Jun. 7, 2012

(54) PROTEINS AND METHOD FOR DETECTION OF LYME DISEASE

(76) Inventors: Carl A. ASCOLI, Pennsburg, PA (US); David P. Chimento, Wynnewood, PA (US); Hiep T. Tran, West Chester, PA (US); Thomas O. Kohl, Pottstown, PA (US)

(21) Appl. No.: 13/310,297

(22) Filed: Dec. 2, 2011

Related U.S. Application Data
(60) Provisional application No. 61/419,028, filed on Dec. 2, 2010.

(51) Int. Cl. G01N 33/569; G01N 33/82; G01N 21/64

(52) U.S. Cl. 435/7.5; 435/7.1; 435/7.9; 435/7.92

(57) ABSTRACT

Recombinant B. burgdorferi proteins, representative of the life cycle, are membrane-immobilized to capture antibodies in biological samples. Lateral flow technology incorporating gold colloid deposition results in band visualization indicative of a positive test.

This invention claims the priority of U.S. Provisional Patent Application No. 61/419,028, filed Dec. 2, 2010, which is incorporated herein by reference.

This invention was made with government support under grant #1R43AI084296-01 awarded by the National Institute of Health. The government has certain rights in the invention.

### FIELD OF THE INVENTION

The invention relates to assays used to test for Lyme disease. More specifically, the invention relates to a composition for testing for Borrelia burgdorferi sensu lato (the causal agent of Lyme disease, including antigens derived from B. burgdorferi, B. garinii and B. afzelii, and other sensu lato Borrelia species, hereinafter B. burgdorferi), an assay for testing for B. burgdorferi using the composition, and a method of testing for Lyme disease. The assay is amenable to use in a lateral flow device and can be used for on-site testing at the point of care. The assay relates to Lyme disease detection in vertebrate animals, including humans, felines, canines, equine and other animal species susceptible to Lyme disease.

### BACKGROUND OF THE INVENTION

Lyme disease (LD) is caused by Gram-negative extracellular spirochetal bacteria from the genus Borrelia. Borrelia burgdorferi sensu stricto is the predominant cause of LD in the United States, whereas Borrelia afzelii and Borrelia garinii are implicated in most Eurasian cases. The term used to collectively describe all three genospecies is Borrelia burgdorferi sensu lato. Borrelia is transmitted to humans by the bite from an infected hard tick belonging to one of several species of the genus Ixodes. In North America the most common transmitter of the disease is the deer tick (Ixodes scapularis). In Europe the transmitter of the disease is called the sheep tick or the castor bean tick (Ixodes ricinus). According to a CDC report, 29,959 confirmed and 8,509 probable cases were reported in the U.S. in 2009, the highest number ever reported. The symptoms of LD in humans occur in three stages. Stage one of LD is often characterized by a distinctive, expanding red rash that usually develops at the site of the tick bite. This rash, known as erythema migrans, is seen in 60-80% of infected individuals. Spirochetes can be isolated from the leading edge of the rash. Erythema migrans is a red circular patch that appears usually 3 days to 1 month following the bite of the tick. The patch then expands, often to a large size and develops a characteristic “bull's eye” appearance. Stage two (dissemination stage) occurs days to weeks following infection. At this stage the spirochetes spread hematogenously to additional body tissues. One or more of the following symptoms and signs may be noted: fatigue, chills and fever, headache, muscle and joint pain, swollen lymph nodes and secondary annular skin lesions. Stage three typically involves intermittent episodes of joint pain. Some symptoms and signs of LD may not appear until weeks, months, or years after a tick bite. Common clinical manifestations at this stage may include meningitis, Bell's palsy, cardiac involvement, and migratory pain to joints, tendons, muscle and bone and chronic arthritis. Other clinical manifestations associated with stage three LD include neurologic complications such as depression, disturbances in memory, mood, or sleep patterns, and sensations of numbness and tingling in the hands or feet.

In most cases, the infection and its symptoms are eliminated with antibiotics, especially if diagnosis and treatment occur early in the course of illness. Late, delayed, or inadequate treatment can lead to late manifestations of LD which can be disabling and difficult to treat. Therefore, early diagnosis and treatment with an appropriate antibiotic is critical.

Diagnosis of LD today predominantly relies on the results of a clinical exam and a history of exposure to endemic LD areas. Serological testing is useful to survey for B. burgdorferi-specific antibodies present in the patient's blood, but is not diagnostic due to the high degree of false positive and false negative error rates for the assays commonly in use today. It is estimated that over 4 million LD serology tests are annually performed in the United States alone. The United States Centers for Disease Control and Prevention (“CDC”) recommends a two-tiered laboratory-based approach when testing blood for evidence of LD, which includes an initial screening test by Enzyme Linked Immunosorbent Assay (“ELISA”) followed by western immunoblot (“western blot”) for determining the presence of B. burgdorferi-specific antibodies.

The Western Blot Approach

A significant limitation of LD western blot testing is the associated variability from laboratory to laboratory and the subjectivity in the visual interpretation of the test strips, requiring a high level of expertise and discretion on the part of the person reviewing the test. Furthermore, western blot testing is time consuming; taking more than 4 hours to perform in a clinical laboratory-based setting.

The western blot-based assay for the detection of B. burgdorferi-specific antibodies has been addressed by a number of independent companies and is currently supplied in the form of ready-to-probe immunoblot strips: These include the MarBlot Lyme IgG (Cat #40-2065G) and the MarBlot Lyme IgM (Cat #40-2065M) from Trinity Biotech (www.trinitybiotech.com, Carlsbad, Calif.) as well as the ViraBlot strips and ViraStripe for detection of human IgM and IgG antibodies (Cat #V-BSBMOK, Cat #V-BSBGOK, Cat #V-BSSGOK and Cat #V-BSSMOK) from Viramed Biotech AG (Planegg, Germany).

The MarBlot and ViraBlot IgM and IgG assays incorporate only the B. burgdorferi strain B31-specific antigens (North American strain) for western blot analysis. In the MarBlot strip, individual antigens are separated by SDS-PAGE electrophoresis, whereas the ViraStripe IgM and IgG assays consist of nitrocellulose membranes onto which ten (10) fractionated and purified B. burgdorferi cell culture lysate-derived proteins have been imprinted at defined locations in equal concentrations.

While both the MarBlot and ViraBlot assays have been approved by the U.S. Food and Drug Administration (FDA) they do not address the following three problems: (i) both use in vitro cell culture-derived B. burgdorferi-specific antigens and as mentioned above, certain B. burgdorferi proteins are only expressed throughout the course of naturally occurring infections. Thus, some cell culture-derived antigens will fail to detect an antibody response to in vivo-specific antigens; these will for example include the B. burgdorferi proteins OspE 14 and the decorin binding proteins (Dbp) A and B which are only predominantly expressed during natural mammalian infection; (ii) whole cell lysates also contain a number of highly conserved housekeeping proteins/antigens, which in turn can be recognized strongly with sera from patients infected with other bacteria. Therefore, there is a potential false positive when whole cell lysates are used; and (iii) both assays rely on western blot technology, are time consuming and require qualified personnel and/or robotic automation.

The whole-cell immunoblot assay encompasses testing for antibodies against a single strain only. The use of several different spirochete strains causing LD would considerably increase the overall costs of routine diagnosis, since every strain would need to be represented on a separate immunoblot strip. Standardization of the whole-cell lysate blot is problematic due to differential expression of immunodominant proteins as well as the discrimination between specific and non-specific antigens. An additional drawback of the LD western blot is that cultured spirochete cell lysates contain numerous highly conserved housekeeping proteins, which antibodies resultant of other bacterial infections may recognize. The presence of these housekeeping proteins potentially could indicate a false positive signal.

While present in a carrying arthropod or an infected mammal (i.e., in vivo), B. burgdorferi undergoes a rapid adaptive gene expression in response to environmental signals encountered during its different life cycle stages. There is, therefore, a varying expression pattern of different proteins in the arthropod vector vs. the mammalian host. These conditions only occur in vivo and cannot be replicated in culture (in vitro). Hence, antibodies generated in response to naturally-occurring proteins will not show a reaction to cell culture-derived spirochete lysates. The absence of a reaction manifests itself as a false negative test result. It has been shown that a number of human antibodies to B. burgdorferi proteins only recognize non-denatured forms; thus the western blot assay which is comprised of denatured proteins will not detect the subset of human antibodies that recognize native epitopes.

Polymerase Chain Reaction

Polymerase chain reaction (“PCR”) has been used to amplify B. burgdorferi genomic DNA in human skin, blood, cerebrospinal fluid and synovial fluid samples. However, the PCR-based assay has not been standardized for routine diagnosis of LD. The accuracy of PCR diagnosis depends on the DNA sample preparation, target and primer selection, the PCR method and the detection system; all of which could affect the overall assay sensitivity and specificity. Also, B. burgdorferi localized in tissue cannot be detected by the use of blood specimen, which limits the utility of PCR testing methods.

A number of independent laboratories have used the PCR-based approach to detect the presence of B. burgdorferi spirochetes in the biological specimen. While PCR, if performed correctly, is a highly sensitive assay for the detection of B. burgdorferi DNA; there are a number of associated technique drawbacks including: (i) the time required for sample preparation, PCR reaction and the required electrophoresis; (ii) the inability of the PCR-based assay to detect the bacterial antigen when such is localized in different body tissues; (iii) the requirement of qualified personnel and expensive equipment; (iv) the direct correlation between sample preparation and achievable results; (v) the non-standardization of the test resulting in result variations between independent laboratories; and (vi) the inability of inter-strain gene amplification using highly strain-specific PCR primers in the event of non-conserved or mutated primer binding regions. The PCR method for LD detection also shows high false negative results.

The POC Idexx SNAP 4Dx Kit

Idexx Laboratories has developed and marketed the POC Idexx SNAP 4Dx kit for detection of LD in animals. This test is based on the antibody detection of the highly specific, conserved and immunodominant C6 region of the B. burgdorferi VlsE surface protein only. The antigen, referred to the C6 peptide, is a 26 amino acid long peptide that reproduces the sequence of the sixth invariable region (IR6) within the central domain of the Borrelia burgdorferi sensu lato VlsE protein. The SNAP 4Dx Test is ELISA-based and involves a washing and an enzymatic-regulated substrate reaction step; both of which contribute to overall assay time and possible background. Published literature indicates that during the course of infection, B. burgdorferi undergoes various stages leading to the fact that not all LD positive patients generate antibodies reactive to VlsE. The use of only one spirochete-specific antigen limits the overall detection capacity of the test and could result in false negative diagnoses. Additional biomarkers are required for full diagnosis of LD. When the C6 test was performed using acute-phase serum samples obtained from >158 characterized patients with erythema migrans burgdorferi, the C6 test for LD showed only 69.5% accuracy. [Wormser, 2008 #1115] Another study found that only 78% of confirmed LD positive samples show antibody reactivity to the C6 epitope. [Wormser, 2008 #1115]

Currently available methods have not achieved a sufficiently high accuracy (i.e., low false negative and low false positive rates relative to the findings of the clinical exam) for the detection of LD. The PCR and immunoblot strip-based approaches remain labor intensive and require qualified personnel to perform the work in a clinical laboratory setting. As mentioned above, the PCR-based approach remains subject to interpretation due to the fact that it is difficult to be standardized. Its prediction accuracy is dependent on sample preparation and when B. burgdorferi spirochetes are localized inside different tissues the bacteria cannot be detected in the blood stream. Furthermore, overall detection accuracy is dependent on primer design and usage. A set of specific primers may amplify the DNA of one specific bacterial strain; however, may not detect a different strain if mutations or rearrangements occur within the primer binding regions.

The immunoblot strips displaying immobilized LD antigen fractions (MarBlot and ViraStripe) do not account for the processing time, the qualified personnel and equipment required for the costly analysis of human biological specimen. Furthermore, these tests rely on LD-specific antigens produced using in vitro cultures and as mentioned above do not reflect the overall protein expression pattern encountered during naturally occurring infections in vertebrate animals. The ViraStripe LD detection assay relies on subjective interpretation by comparison of overall banding pattern to the reactivity of an assay-internal control band.

While the development of the Idexx 4Dx Snap Assay has significantly reduced the biological sample assay time to only eight (8) minutes, the incorporation of only the C6 epitope as the antibody-detection antigen falls short of correctly determining the presence of LD-specific antibodies in otherwise characterized LD positive samples.

In summary, the currently applied methods for the detection of B. burgdorferi (LD) are not only labor intensive, time-consuming and expensive; they require qualified personnel working in a specialized laboratory setting. These assay requirements are not suitable for the Point-of-Care (“POC”) application in areas of the world where qualified laboratory personnel might be in short supply, including rural areas where LD might be prevalent. In addition, the requirements of qualified personnel, specialized laboratories and a time intensive process make the detection of LD by the existing methods an expensive proposition, and therefore, these tests are not economically feasible for the poor and uninsured. Increases in health care costs result in a greater number of people without health insurance, which may limit the accessibility and affordability of regular tests for LD for many individuals.

There is a need for a test for LD that is cost-effective, time-efficient, can be performed on varying types of bodily fluids, and can accurately detect LD despite the mutations and other changes during the life cycle of B. burgdorferi.

### SUMMARY OF THE INVENTION

An object of the present invention provides compositions for the detection of Lyme disease (LD). The composition contains at least two of the following proteins: OspA (SEQ ID NO: 1), OspB (SEQ ID NO: 2), OspC (SEQ ID NO: 3), OspE (SEQ ID NO: 4), VlsE (SEQ ID NO: 5), CRASP-1 (SEQ ID NO: 6), CRASP-2 (SEQ ID NO: 7), DbpA (SEQ ID NO: 8), DbpB (SEQ ID NO: 9), Flagellin (SEQ ID NO: 10), Arp37 (SEQ ID NO: 11), P27 (SEQ ID NO: 12), P35 (SEQ ID NO: 13), P39 (SEQ ID NO: 14), P66 (SEQ ID NO: 15), BBK32 (SEQ ID NO: 16), and RevA (SEQ ID NO: 17). The proteins are preferably purified full length recombinant proteins that are identical to Borrelia proteins present in vivo post-infection and during chronic infection. Preferably, the composition contains at least two of the following proteins: OspA (SEQ ID NO: 1), OspB (SEQ ID NO: 2), OspC (SEQ ID NO: 3), OspE (SEQ ID NO: 4), VlsE (SEQ ID NO: 5), CRASP-1 (SEQ ID NO: 6), CRASP-2 (SEQ ID NO: 7), DbpA (SEQ ID NO: 8), DbpB (SEQ ID NO: 9), Arp37 (SEQ ID NO: 11), P27 (SEQ ID NO: 12), P35 (SEQ ID NO: 13), P39 (SEQ ID NO: 14), P66 (SEQ ID NO: 15), BBK32 (SEQ ID NO: 16), and RevA (SEQ ID NO: 17). In preferred embodiments, the composition contains at least four, at least six, at least ten, or at least thirteen of the proteins. A preferred composition contains Arp 37, OspE, p39, OspC, VslE, DbpB, OspB, DbpA, p35, OspA, Crasp-2, p27 and Crasp-1.

Another object of the present invention relates to methods for using the composition to detect the presence of antibodies against B. burgdorferi in the biological fluid of an individual (human or animal). The method contains contacting the individual's biological fluid with the composition. In the presence of B. burgdorferi infection of the individual, an antigen-antibody complex is formed by the binding of the protein(s) of the composition with antibodies. Subsequently, the reaction mixture is analyzed to determine the presence or absence of these antigen-antibody complexes. Because B. burgdorferi is the causative agent for LD, this method is also useful to detect and diagnose LD. In a preferred embodiment, antibody complex with at least two proteins of the composition indicates the presence of LD. In another preferred embodiment, serum is used as the biological fluid. The methods can be adapted for use with immunoassays known in the art, e.g. enzyme linked immunosorbent assay (ELISA) and immunofluorescence assay (IFA).

A further object of the present invention provides test strips to detect the presence of B. burgdorferi or to detect and diagnose LD. The test strip contains at least two regions, each of which containing a protein selected from OspA (SEQ ID NO: 1), OspB (SEQ ID NO: 2), OspC (SEQ ID NO: 3), OspE (SEQ ID NO: 4), VlsE (SEQ ID NO: 5), CRASP-1 (SEQ ID NO: 6), CRASP-2 (SEQ ID NO: 7), DbpA (SEQ ID NO: 8), DbpB (SEQ ID NO: 9), Flagellin (SEQ ID NO: 10), Arp37 (SEQ ID NO: 11), P27 (SEQ ID NO: 12), P35 (SEQ ID NO: 13), P39 (SEQ ID NO: 14), P66 (SEQ ID NO: 15), BBK32 (SEQ ID NO: 16), and RevA (SEQ ID NO: 17).

A yet further object of the present invention provides test kits for detecting B. burgdorferi or diagnosing LD. The kit contains at least the composition of the present invention. Other components that may be included in the kit include antibodies specific against the proteins of the composition, diluents (e.g. buffer solutions), and instruction for performing the test.

### BRIEF DESCRIPTION OF THE DRAWINGS

FIG. 1 is a representative figure showing fourteen recombinant Lyme Disease proteins, some or all of which are used in the preferred embodiment of the present invention.

FIG. 2 is an illustration view of the portions of a lateral flow test strip for use in the preferred embodiment of the present invention.

FIG. 3 is an exploded view of the sample pad of the lateral flow test strip for use in the preferred embodiment of the present invention.

FIG. 4 is an exploded view of the gold-conjugate pad of the lateral flow test strip for use in the preferred embodiment of the present invention.

FIG. 5 is an exploded view of the nitrocellulose membrane of the lateral flow test strip for use in the preferred embodiment of the present invention.

FIG. 6 is a schematic of a four (4) window cassette for B. burgdorferi-specific IgG or IgM antibody detection in accordance with the preferred embodiment of the present invention.

FIG. 7 is a representative figure showing the results of one of two test strips in accordance with a preferred embodiment of the present invention.

FIG. 8 is a schematic presentation of the radial multi-directional flow device. The central sample pad is impregnated with goat anti-human IgM or IgG for the retention of respective serum-based immunoglobulins. LD serum samples will be diluted in sample buffer and applied to the sample pad. The antibody/antigen complex flows outward until the complex recognizes and binds the specific immobilized antigen resulting in gold deposition for visualization by the user. Visualization of a flow control band validates the performance of the assay.

FIG. 9 is a photograph of a gel showing IPTG-induced expression of 14 recombinant B. burgdorferi fusion proteins. Over-expression of the fusion proteins is indicated by the presence of a band not reflected in the control.

FIG. 10 are photographs of gels showing MBP-OspE purification shown in panels (A) Elution of the MBP-OspE protein from repeated binding steps to Amylose resin. (B) TEV protease-mediated cleavage of the fusion protein, thus releasing the recombinant protein from the MBP. (C) Fraction analysis following MBP and OspE protein separation by SEC.

FIG. 11 is a photograph showing Western blot of LD antigens Osp C, Crasp-2 and p39. LD antigens were expressed as FLAG-fusion proteins in E. coli and were probed separately with corresponding anti-LD antibodies. Arrows indicate the expected molecular weight of each antigen.

FIG. 12 are photographs of test strips showing IgG and IgM differentiating tests showing individual serum reactivity and band visualization patterns of imprinted B. burgdorferi antigens, in the order shown. Diluted serum samples were premixed with liquid gold-labeled recombinant LD proteins prior to application onto the sample pad. The flow control band closest to the wick on strips encompassing antigens 1-7, indicates proper performance of the test. No reactivity of the MBP negative control band (imprinted closest to the wick containing antigens 8-14) is observed in any of the human sera.

FIG. 13 are photographs showing polyclonal antibody reactivity to specific membrane immobilized B. burgdorferi proteins in the order shown to the left.

FIG. 14 is a table showing data obtained from the evaluation of clinically confirmed LD positive sera (n=20) and LD negative control sera (n=5). None of the sera from individual donors show reactivity to the MBP control. All 5 LD negative control sera did not react with any of 14 B. burgdorferi antigens in both the IgG and IgM assays.

FIG. 15 are pie charts showing that the present invention eliminates false positive results. The accuracy of the present invention is compared with the accuracy of the C₆ VlsE ELISA test. The present invention correctly identifies 9 samples as LD negative whereas the currently used C₆ VlsE ELISA test identified these samples as false positive when compared with western blot results and clinical findings.

### DETAILED DESCRIPTION OF THE PREFERRED EMBODIMENTS

The present invention described herein demonstrates seventeen (17) recombinant proteins that are detected by antibodies present in serum samples of patients diagnosed with Lyme disease. B. burgdorferi genomic DNA and gene specific primers for PCR-based amplification were used to produce seventeen (17) B. burgdorferi-specific proteins representative of the in vivo life cycle of the bacteria. Cloned genes were inserted by ligation independent cloning into fusion protein expression vectors for transformation of competent E. coli cells. High fusion protein-expressing clones were grown in bacterial cultures and after induction the B. burgdorferi proteins were isolated and purified using standard laboratory methods. Other suitable methods for making recombinant proteins are known in the art and are within the skill of a person in the art.

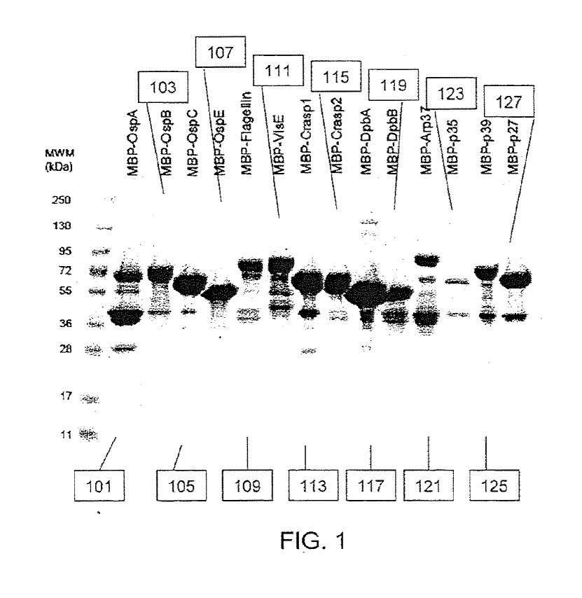

FIG. 1 shows fourteen MBP-Borrelia fusion proteins by representations of their molecular weight. As discussed in further detail below, a complex of some or all of the 17 proteins with antibodies in an individual's biological fluid indicates that the individual is infected with Lyme disease (LD). In an embodiment, LD is diagnosed in an individual if at least two of the 17 proteins form complex with antibodies in the individual's biological fluid, preferably at least six of the 17 proteins, more preferably at least ten of the 17 proteins, and most preferably all of the 17 proteins.

An Outer Surface Protein A (“OspA”) protein 101 is shown. OspA 101 has a UniProt Accession Number of P0C926. Development of antibodies against OspA 101 occurs in the early stages of infection, toward the beginning of prolonged arthritis episodes. OspA-specific antibodies also display cross-reactivity with leukocyte function associated antigens (LFA) [Trollmo et al., 2001]. IgG and IgM antibodies to OspA 101, OspB 103 and OspC 105 (hereinafter described) are widely presented in EM patient sera. High expression levels and purification of a soluble recombinant form of OspA 101 have been obtained. OspA 101 has a molecular weight MBP-Fusion of 70.5 kDa and a molecular weight Borrelia protein of 28.1 kDa.

An Outer Surface Protein B (“OspB”) protein 103 is shown. OspB 103 has a UniProt Accession Number of P17739. OspB 103 is involved in the transmission and establishment of early infections. OspB 103 is a surface protein highly specific to Borrelia and acts a marker for early infections. Escherichia coli-based expression of recombinant full-length outer surface proteins A (OspA 101) and B (OspB 103) have been used to develop the LD vaccine for use in animals. OspB 103 has a molecular weight MBP-Fusion of 72.7 kDa and a molecular weight Borrelia protein of 30.3 kDa.

An Outer Surface Protein C (“OspC”) protein 105 is shown. OspC 105 has a UniProt Accession Number of Q07337. OspC 105 is involved in the transmission and establishment of early infections. OspC 105 is a surface protein highly specific to Borrelia and acts as a marker for early infections. IgM and IgG antibodies to OspA 101, OspB 103 and OspC 105 are widely encountered in EM patient sera. A high expression yield of recombinant OspC 105 has been obtained in E. coli. Significant improvements of the recombinant Borrelia-specific IgG immunoblot test have been achieved by the addition of the VlsE, DbpA and OspC proteins. OspC 105 has a molecular weight MBP-Fusion of 63.1 kDa and a molecular weight Borrelia protein of 20.7 kDa.

An Outer Surface Protein E (“OspE”) protein 107 is shown. OspE 107 has a UniProt Accession Number of C0R6T1. OspE 107 is involved in early and middle stage Lyme Disease infections. OspE 107 is a member of the paralog family of erp genes and has evolved from a common ancestral gene by speciation. It has been demonstrated that many Erp proteins bind to the host complement regulator factor H and help to protect bacteria from complement-mediated killing during mammalian infection. Erp genes are detectably transcribed in various tissues three months or more post infection. OspE 107 is expressed in mammals and elicits a strong antibody response. Furthermore, OspE 107 has been expressed and purified using the T7 expression system. OspE 107 has a molecular weight MBP-Fusion of 59.5 kDa and a molecular weight Borrelia protein of 17.1 kDa.

A Flagellin 109 is shown. Flagellin 109 has a UniProt Accession Number of COALE1. Flagellin 109 is present in early, middle and late stages of infection. Flagellin 109 is a 41 kDa antigen which has been used as a biomarker in the Lyme Disease immunoblot assay recommended by the CDC. Flagellin 109 has been successfully expressed and purified from E. coli. Flagellin 109 has a Molecular weight MBP-Fusion of 76.3 kDa and a Molecular weight Borrelia protein of 33.9 kDa.

A VlsE 111 is shown. VleE has a UniProt Accession Number of Q5DVG3, and is present in the middle and late stages of infection. VlsE 111 is a 35 kDa lipoprotein that undergoes antigenic variation by gene conversion with a silent vls cassette and plays a major role in the immune response. The VlsE protein 111 has been expressed and purified from E. coli. Significant improvements of the recombinant Borrelia-specific IgG immunoblot test have been achieved by addition of VlsE 111, DbpA and the OspC 105 proteins from Borrelia. VISE 111 has a molecular weight MBP-Fusion of 78.7 kDa and a molecular weight Borrelia protein of 36.3 kDa.

A Complement regulator-acquiring surface protein-1 (CRASP-1) 113 is shown. CRASP-1 113 has a UniProt Accession Number of Q66ZC1. CRASP-1 113 is present in the middle and late stages of infection. CRASP-1 along with CRASP-2, -3, -4, -5 and the Erp proteins contribute to spirochete invasion and survival in humans by binding to the serum factor H. The 26 kDa B. burgdorferi CRASP-1 protein is a dominant factor H and FHL-1 binding protein. It is expressed in humans and induces antibody responses that are restricted to non-denatured structural determinants. CRASP-1 113 has a molecular weight MBP-Fusion of 69.3 kDa and a molecular weight Borrelia protein of 26.9 kDa.

A Complement regulator-acquiring surface protein-2 (CRASP-2) 115 is shown. CRASP-2 115 has a UniProt Accession Number of O50665. CRASP-2 115 is present in the middle and late stages of infection, and is a serological marker for human Lyme disease. CRASP-1 113 and CRASP-2 115 have been expressed in E. coli and recent studies have established that CRASP-2 is ubiquitously expressed in mammals and elicit a robust antibody-mediated response in infected hosts including humans, but not in ticks. CRASP-2 115 has a molecular weight MBP-Fusion of 67.8 kDa and a molecular weight Borrelia protein of 25.4 kDa.

A Decorin Binding Protein A (DbpA) 117 is shown. DbpA 117 has a UniProt Accession Number of O50917. DbpA 117 is present in the late stage of infection. Spirochaetal surface adhesion is mediated by attachment to decorin, a major component of the host extracellular matrix. This enables bacteria to colonize mammalian tissues. Significant improvements of the recombinant Borrelia-specific IgG immunoblot test have been achieved by addition of VlsE 111, DbpA 117 and the OspC 105 proteins from Borrelia. Both B. burgdorferi DbpA 117 and DbpB 119 proteins lacking the hydrophobic N-terminus have been expressed and purified from E. coli. DbpA 117 has a molecular weight MBP-Fusion of 60.9 kDa and a molecular weight Borrelia protein of 18.5 kDa.

A Decorin Binding Protein B (DbpB) 119 is shown. DbpB 119 has a UniProt Accession Number of O50918. DbpB 119 is present in the late stage of infection. Antibodies to DbpA 117 and DbpB 119 are predominantly present in the cerebrospinal fluid (CSF) and serum samples from patients with confirmed neuroborreliosis as well as patients with suspected neuroborreliosis. Both B. burgdorferi DbpA 117 and DbpB 119 proteins lacking the hydrophobic N-terminus have been expressed and purified from E. coli. DbpB has a molecular weight MBP-Fusion of 60.3 kDa and a molecular weight Borrelia protein of 17.9 kDa.

An Arthritis-related protein Protein (Arp37) 121 is shown. Arp37 121 has a UniProt Accession Number of O51011, and is present in the late stage of infection. Approximately 60 to 80% of study patients with Lyme Disease have shown an IgG antibody response to the 37 kDa arthritis-related protein (Arp) of B. burgdorferi. Recombinant Arp37 protein has been expressed and purified from E. coli. Arp37 121 has a molecular weight MBP-Fusion of 78 kDa and a molecular weight Borrelia protein of 35.6 kDa.

A p35 protein 123 is shown. P35 123 has a UniProt Accession Number of O50687. P35 123 is present in the middle and late stages of infection, and is selectively expressed in vivo and induces a strong immune response. 72% of sera collected from patients with Lyme borreliosis developed anti-p35 antibodies. P35/bba64 expression is highly upregulated in the bladder, heart and spleen tissues throughout the infection period in murine models. P35 123 has a molecular weight MBP-Fusion of 69.5 kDa and a molecular weight Borrelia protein of 27.1 kDa.

The immunodominant antigen p39 (bmpA) (“P39”) 125 is shown. P39 has a UniProt Accession Number of Q45010. P39 is present in the late stages of infection. Basic membrane proteins A and B (bmpA; bmpB) are preferentially expressed in joint tissues of infected animals. B. burgdorferi lacking the bmpA/B genes were infectious in mice, but unable to persist in joints, thus failing to induce severe arthritis. Antibodies to bmpA and bmpB have been shown in Lyme Disease patients. P39 has a molecular weight MBP-Fusion of 77.8 kDa and a molecular weight Borrelia protein of 35.4 kDa.

A 27 kDa surface lipoprotein (“P27”) 127 is shown. P27 127 has a UniProt Accession Number of O50951, and is present in the Early, Mid and Late stages of infection. The 27 kDa surface lipoprotein gene has been cloned, expressed and purified from E. coli [Reindl et al., 1993]. A BLAST alignment revealed that this protein is highly conserved in all Borrelia species, displaying no homology to proteins from the other genus. P27 127 has a molecular weight MBP-Fusion of 73.3 kDa and a molecular weight Borrelia protein of 30.9 kDa.

P66 is an adhesion, the ligand for the beta (3)-chain integrin, expressed by B. burgdorferi in mammals. P66 was recognized strongly with sera from patients in early and late states. Native P66 protein has a molecular weight of 68.2 kDa.

Fibronectin binding protein, BBK32 is strongly recognized with sera from patient in various state with sensitivity ranging from 70 to 100%. BBK32 has been expressed and purified from E. coli as GST fusion. Native BBK32 protein has a molecular weight of 41.6 kDa.

Fibronectin-binding outer surface protein, RevA, is expressed early in human infection and recognized with a sizable proportion serum samples from North America and European patients in initial stages. Native RevA protein has a molecular weight of 17.9 kDa.

Potentially all of the above 17 proteins may be detected by antibodies present in serum samples from humans or animals diagnosed with LD. As one of skill in the art would recognize, the test can be effective without inclusion of each individual protein of the 17 proteins, and when a test is administered, all proteins (whether 17 proteins or fewer are included in the test) do not need to be detected in order for a positive test result to be recorded. In an embodiment, at least one protein, preferably at least two, present in the early stage of infection (OspA, OspB, OspC, OspE, p27, p66, BBK32 and RevA), at least one, preferably at least two, protein present in the middle stage of infection (OspE, Flagellin, VlsE, CRASP-1, CRASP-2, p35, p27 and BBK32), and at least one, preferably at least two, protein from the late stage of infection (Flagellin, VlsE, CRASP-1, CRASP-2, p35, p27, BBK32, DbpA, DbpB, Arp37, p39 and p66) would be included in the assay for detection of B. burgdorferi. For further testing accuracy, it may be preferable to include, four, five, six, or seven of the 17 proteins. Proteins that are present during the early stage of the infection are the most critical biomarkers for a timely diagnosis of the disease. In a preferred embodiment at least four of the 17 proteins are used to positively diagnose and detect LD.

Due to the presence of the proteins involved in the early, middle, and late stage of the infection, the present invention is particularly useful in identifying the stage of infection of an individual. This allows the treating physician the information needed to formulate a treatment regime as different stages of infection require different treatment regimens. As such, the present invention can also be used to detect early stage infection by including at least one, preferably at least two, of the following proteins in the composition: OspA, OspB, OspC, OspE, p27, p66, BBK32 and RevA. Likewise, middle stage infection requires at least one, preferably at least two, of the following proteins in the composition: OspE, Flagellin, VlsE, CRASP-1, CRASP-2, p35, p27 and BBK32; and late stage infection requires at least one, preferably at least two, of the following proteins: Flagellin, VlsE, CRASP-1, CRASP-2, p35, p27, BBK32, DbpA, DbpB, Arp37, p39 and p66. Because some proteins overlap between the stages, to distinguish one stage from another at least one of the non-overlapping proteins is preferably used. For example, OspE is in the early and the middle stages. As such, to distinguish those two stages at least one other protein is required. Thus, positive reactions for OspE and, e.g. OspA, indicate early stage infection, while positive reactions for OspE and, e.g. VlsE indicate middle stage infection. The ability to distinguish one stage of infection from another is important in assisting the treating physician to devise a treatment regimen.

The protein-antibody complexes can be detected using immunoassay techniques known in the art. Generally, immunoassays involve the binding of the proteins and antibodies in the individual's biological fluid. The presence of binding indicates that the individual is infected with B. burgdorferi. Examples of immunoassays include, but are not limited to, ELISAs, radioimmunoassays, and immunoblots, which are well known in the art. The labels used to detect the protein-antibody complexes can be, but are not limited to biotin, fluorescent molecules, radioactive molecules, chromogenic substrates, chemi-luminescence, and enzymes.

In an embodiment, ELISA, based on the capture of the antibodies by immobilized proteins, followed by detection with anti-IgG or anti-IgM antibody, is used to detect anti-B. burgdorferi antibodies in the individual's biological fluid. In this system, each well of a multi-well plate is coated with one of the 17 proteins. The biological fluid are then added to the wells and incubated for capture of the antibodies (if present) by the proteins. The plate is then detected with, e.g. fluorescein conjugated anti-IgG antibody.

Although persons skilled in the art will realize the applicability of the 17 protein testing assay in other testing platforms, a preferred embodiment of the present invention uses a test strip, which is well known in the art. In general, the test strip contains a sample pad where a biological fluid sample is placed. The fluid is then wicked along a flow path through a labeling zone where the anti-B. burgdorferi antibodies in the biological fluid are labeled, then a capture/visualization zone where the same antibodies are captured and visualized to detect or diagnose B. burgdorferi infection, and thus, LD. Examples of test strips that may be appropriate for the present invention include, e.g., in U.S. Pat. Nos. 7,569,397; 7,132,078; 6,824,975; 6,620,626; 6,528,325; and 5,447,837; and U.S. Patent Application Publication Nos. 2008/0254444 and 2007/0128587; all of which are incorporated herein by reference.

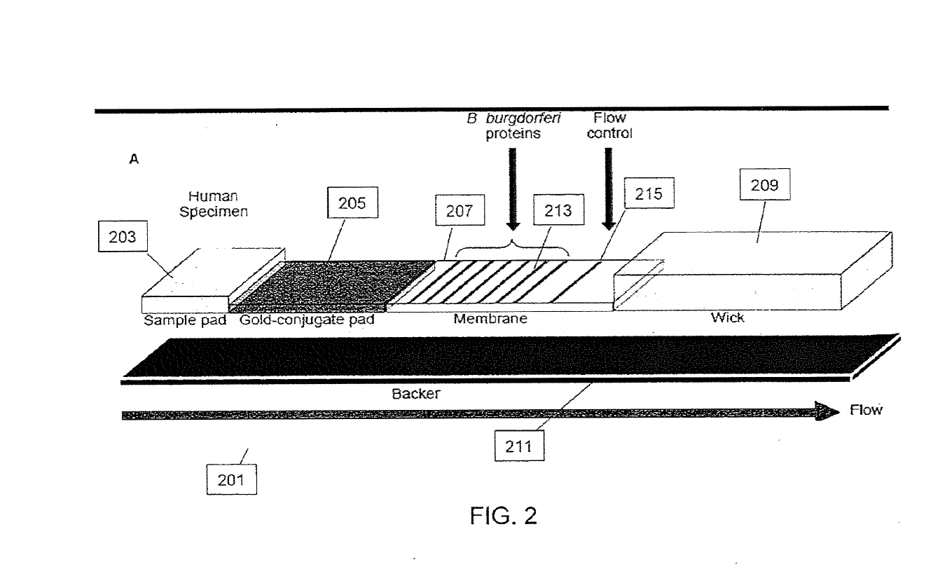

In an embodiment, a lateral test strip can be used for the present invention. Referring to FIG. 2, an illustration view of the portions of the lateral flow test strip 201 for use in an embodiment of the present invention is shown. Lateral flow immunochromatographic assays are well known in the art and are used for applications such as home pregnancy tests. The lateral flow strip comprises a sample pad 203, a gold-conjugate (label) pad 205, a membrane 207, a wick 209, and a backer 211. The membrane (e.g. nitrocellulose) contains protein bands 213 and a flow control band 215. Individual test strip components as depicted in the illustration are layered onto the backer 211 in the order shown. The sample pad 203 is exposed for sample application and the membrane 207 is covered with a transparent window for visualization. A plastic case (not shown) will enclose the assembled device.

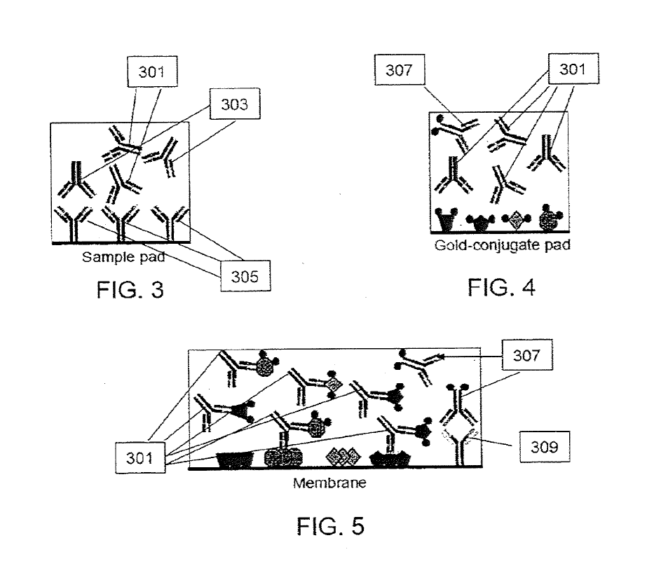

Turning to FIG. 3, prior to specimen application onto the sample pad 203, biological samples are diluted in a sample application buffer. Lateral flow test strips are designed to differentiate in the analysis between IgG or IgM antibody-mediated immune responses against B. burgdorferi. Specifically, the sample pad 203 consists of absorbent material and is impregnated with species-specific anti-IgG Fc 305 or anti-IgM Fc 305 antibodies that retain respective counterparts present in the applied diluted serum (i.e. either IgG 301 or IgM 303) and acts as a filter prior to sample movement onto the gold-conjugate pad. The sample pad is impregnated with species-specific anti-IgG Fc or IgM Fc 5 antibodies 305. Directly applied diluted human serum is separated into either LD reactive IgG or IgM antibodies prior to continued flow onto the gold-conjugate pad.

Turning now to FIG. 4, differentiated anti-B. burgdorferi antibodies contained in the biological serum sample (i.e. only antigen-specific IgG or IgM) flows from the sample pad 203 onto the gold-conjugate pad 205. This pad consists of absorbent material which has been impregnated with at least four (4) gold-conjugated B. burgdorferi-specific proteins. Upon rehydration of the gold conjugate pad 205, the anti-B. burgdorferi antibodies are allowed to bind the gold-labeled recombinant proteins through recognition of one antibody/antigen-specific binding domain 301 prior to flow of the antibody/recombinant protein complex across the imprinted nitrocellulose membrane. Rehydration of the gold-conjugate pad 205 also facilitates the flow of the gold-labeled flow control antibodies 307 across the imprinted nitrocellulose membrane 207. The gold-conjugate pad 205 is impregnated with gold-labeled recombinant B. burgdorferi-specific proteins which upon rehydration of the pad 205 bind to the antigen-specific antibodies in the human serum, prior to flowing across the imprinted membrane 207. Hydration of the sample pad facilitates the movement of flow control IgG or IgM 307 antibodies to flow across the membrane, forming part of the internal flow control.

Turning now to FIG. 5 and also referring to FIG. 2, the nitrocellulose membrane 207 has been immobilized with at least four of the B. burgdorferi-specific proteins 213 identical to those present in the gold-conjugate pad 205; however, these recombinant proteins have not been gold-labeled (as shown in FIG. 4). Preferably, each of the proteins is immobilized in a separate portion of the membrane 207, so that the particular protein can be determined by its specific location (each band 213 on the membrane 207 illustrates a separate protein). The antibody/gold-labeled antigen complex flows across the membrane until the complex encounters its specific imprinted recombinant protein. Through binding of the antibody/gold-labeled antigen complex to the imprinted protein, facilitated by the second antibody/antigen-specific binding domain, deposition of the gold results in the visualization of a band. The membrane comprises species-specific IgG antibodies 301, gold-conjugated flow control antibodies 307 and at least one immobilized antibody 309. The flow control antibody can be, e.g. anti-Human IgG F(c) for the IgG panel and anti-Human IgM Fc5μ for the IgM panel.

Similarly, the rehydrated gold-labeled flow control antibody flows across the membrane until it encounters its imprinted counterpart at the end of the membrane, upon which binding of the antibodies results in gold deposition and is indicative of a test in which the flow across the membrane has been properly performed.

The length of the nitrocellulose membrane 207 of FIG. 2 determines the efficient flow of the biological specimen across the membrane. To ensure proper flow across the pre-blocked membrane resulting in visualization of the flow control band 215, it is preferred that the imprinting of recombinant B. burgdorferi-specific proteins onto the membrane is limited to less than six or seven (7) of the seventeen (17) different recombinant B. burgdorferi-specific proteins. Thus, to test all seventeen (17) proteins, preferably thirteen (13) proteins, multiple imprinted membranes can be used.

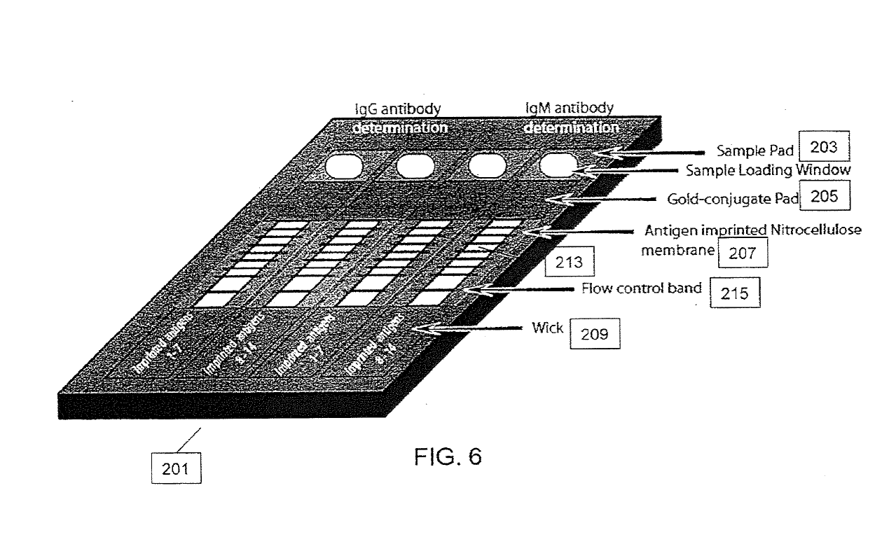

A comprehensive test format may be as illustrated in FIG. 6, in which an anti-B. burgdorferi-specific IgG test will consist of two membranes 207, each with their own sample loading pad. Similarly, the same will hold true for the anti-B. burgdorferi-specific IgM test and both will be contained in the four (4) window cassette as illustrated in FIG. 6.

Turning back to FIG. 2, the wick 209 consists of ultra-absorbent material and facilitates the continued regulated flow of the antibody/gold-labeled antigen complex across the membrane 207 prior to gold deposition at membrane imprinted complementary antigens. Any excess buffer is completely absorbed by the wick 209.

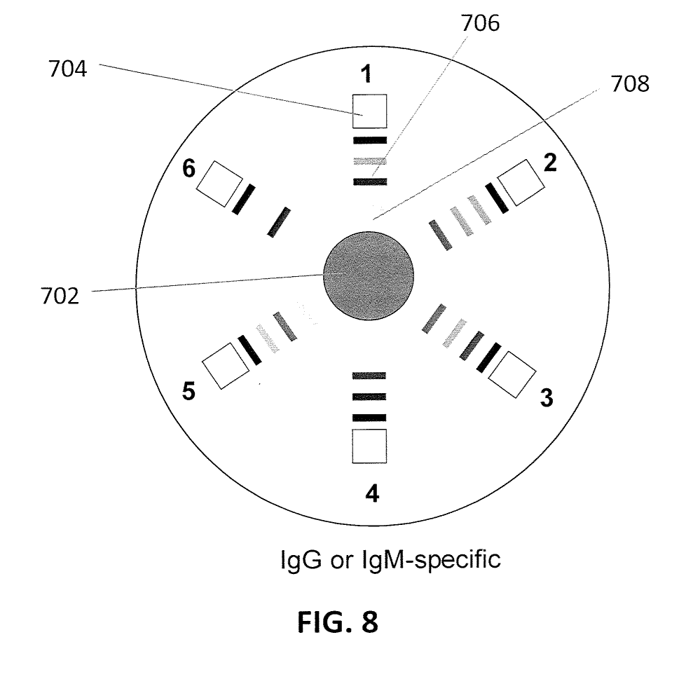

As an alternative to the multiple-parallel test strip device illustrated in FIG. 6, a radial multi-directional flow device can be used, as illustrated in FIG. 8. Here, a single sample pad 702 is located at the center, with flow radiating outward from the center. The biological fluid first flows through a gold conjugate pad 708, and an antigen imprinted membrane 706. Each radial strip also contains a wick 704 at the end. This embodiment is similar to that illustrated in FIG. 6 except that the test strips are arranged radially rather than in parallel fashion. Each of the sample pad 702, the gold conjugate pad 708, the antigen imprinted membrane 706, and the wick 704 is contain the same components as the sample pad 203, the gold conjugate pad 205, the antigen imprinted membrane 207, and the wick 209 described earlier with respect to FIG. 2.

This lateral flow assay using a combination of some or all of the seventeen selected proteins presents many advantages over the prior art. It does not require complex sample processing, because there is no genomic DNA extraction, PCR, or complex application-specific equipment-dependent protocols ViraStripe and Marblot. Its broad spectrum of bacterial antigens covering up to seventeen individual proteins as expressed throughout naturally occurring infections ensure the detection by antibodies present within biological specimen of infected individuals. Gold-deposition results in the accurate development of indicated band intensity and correlates directly with the presence of LD-specific antibodies present in biological specimen. It is not subject to interpretation by comparison of individual band intensities to the intensity of an internal control band (e.g., ViraStripe) for the confirmation of LD. It differentiates by immunoglobulin type, allowing for determination of both the IgG and IgM antibody-mediated immune response (acute and latent stage of infection). It is presented in the form of a single four (4) window format cassette (see FIG. 6) allowing for both the IgG and IgM-specific tests to be performed simultaneously devoid of intra-assay variation. Specific results become available within 25 to 30 minutes after application of the biological specimen.

To use the lateral assay of the preferred embodiment of the present invention, the user applies a sample of bodily fluid to the test onto the sample pad 203. In a preferred embodiment, the sample is blood. However, one of ordinary skill in the art would recognize the ability to use other bodily fluids where the antibodies to the LD proteins would be present. The sample is adsorbed onto the pad 203 and antibodies present in the sample flow towards the wick 209 by capillary action. Binding of antibodies present in the sample to antigens bound to colloidal gold occurs and form a complex that continue to flow until the complex binds antigens immobilized on the membrane 207. After the flow reaches the flow control strip 215 the user evaluates the bands that appear against the protocol to determine test results.

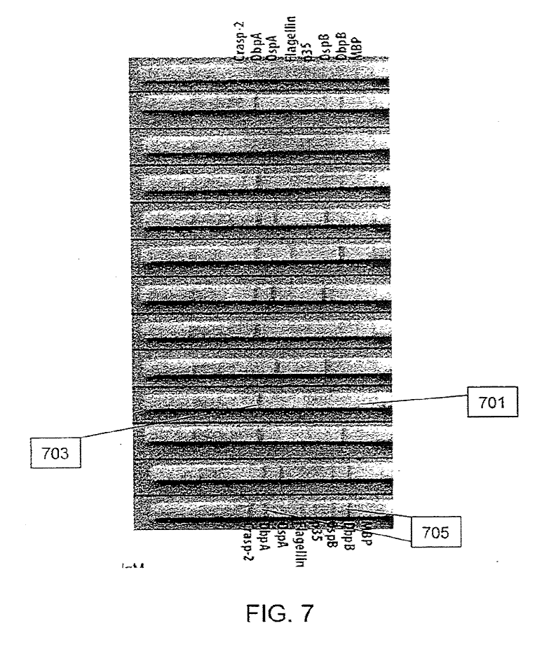

FIG. 7 is a representative figure showing a test strip in accordance with a preferred embodiment of the present invention. Thirteen bands 701 are shown. The bands are mostly light in color, with the exception of darker strips 703 which are shown in seven different horizontal locations 705 on each of the bands 701, with varying degrees of darkness. Each horizontal location 705 (the reference numeral pointing to two of the seven horizontal locations 705) corresponds to one of the seven proteins (of the seventeen total proteins discussed above) that are detected by the test in accordance with a preferred embodiment. The presence of a darker strip 703 in a band 701 at a location 705 indicates that antibodies for the protein associated with that horizontal location 705 are present in the sample. The intensity of the dark strip 703 correlates to the level or amount of the protein in the sample. Reading and evaluating the test results on a strip such as the strip depicted in FIG. 7 requires a lower level of skill than prior LD testing methods, and reduces the subjectivity required to evaluate the test in comparison to prior methods.

While assays commonly in use today for the serological survey of LD antibodies present in the sera of patients diagnosed with LD are useful, the relatively high percentages of false negative and false positive test results for these assays renders their use unsuitable for diagnosis as defined by the CDC. In preliminary comparison studies the test of the preferred embodiment of the present invention yielded positive test results for samples from patients diagnosed with LD when the assays commonly in use today registered false negative results. The test of the present invention is able to identify early stage and late stage LD with a similar low false negative rate. Samples weakly reactive in the commercially available quantitative ELISA test showed greater reactivity in the more sensitive test of the preferred embodiment in comparison studies. In addition, the test of the preferred embodiment requires less skill by the user to perform and analyze. The present invention differentiates early/acute and late/chronic stages of LD relative to IgM and IgG detection, respectively.

Immobilized Borrelia antigens on a solid surface are used to capture respective Borrelia-specific antibodies in bodily fluids. In the preferred embodiment of the present invention, Borrelia-specific antibodies are first bound to Borrelia antigens conjugated to colloidal gold particles. This antibody/antigen complex flows across the membrane and binds specific antigens immobilized on a membrane, resulting in gold deposition and subsequent visualization by the user. A person skilled in the art can use different sensors to detect the bound antibodies other than colloidal gold. Examples include the enzyme horseradish peroxidase or other enzymes or fluorescent or luminescent reporter molecules. Bound Borrelia antibodies can be detected using a variety of methods including electromagnetic and optical sensors including those developed by Axela Biosensors, Biacore, Luminex, Quantum Dot, MesoScale and others. Additionally, a person skilled in the art will recognize that antigens can be immobilized on microchips or other nanotechnologies.

Persons having skill in the art will recognize the utility of the present invention for LD assays for all vertebrate animals susceptible to infection by Borrelia by minor alteration of assay components. Thus, there is veterinary application potential. The assay can be modified to incorporate recombinant Borrelia-specific proteins specific to the Eurasian spirochete species and other species identified as B. burgdorferi sensu lato. It is cost effective, laboratory-independent and can be used by non-professionals in the field at the point-of-care site. It is also cold-chain independent.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### EXAMPLE 1

### Selection, Design and Production of Antigens for Borrelia-Specific Antibody Detection

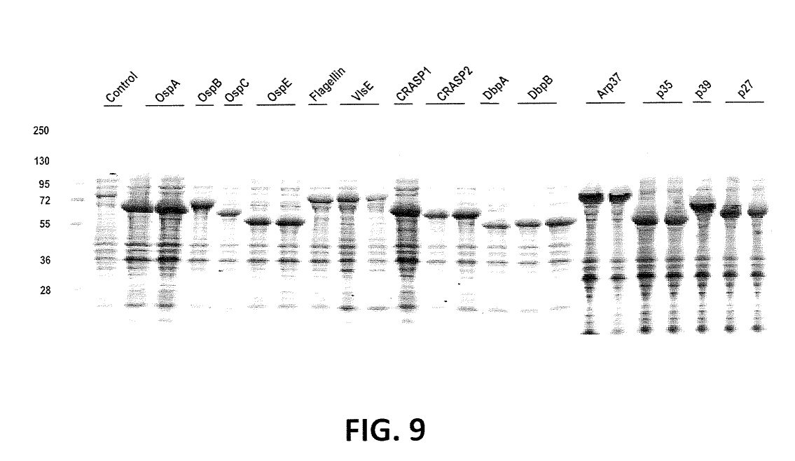

B. burgdorferi genomic DNA (Catalog #35210D-5) was obtained from the ATCC and fourteen (14) B. burgdorferi-specific proteins representative of the in vivo life cycle post infection: OspA, OspB, OspC, OspE, Flagellin, VlsE, Crasp-1, Crasp-2,DbpA, DbpB, Arp37, p35, p39 and p27 were selected to be included in the assay design. Gene-specific primers for the PCR-based amplification of individual bacterial genes were designed and synthesized. Amplified genes were inserted into a modified pMa1C2X vector (NEB #E8200S, New England Biolabs) by ligation independent cloning. Cloned inserts were verified by DNA sequencing and confirmed vectors were transformed into the E. coli expression strain BL21(DE3)pLysS. All fourteen MBP/B. burgdorferi fusion proteins were successfully expressed in E. coli upon IPTG induction (FIG. 9).

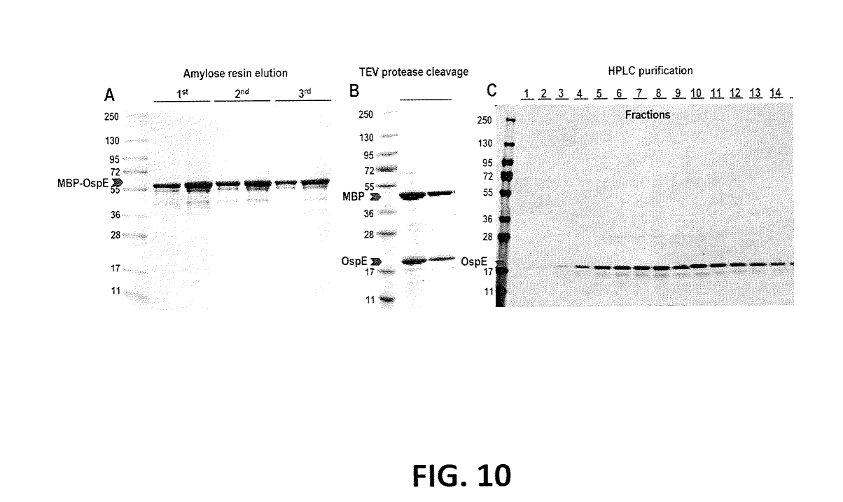

Cells from induced two liter cultures were harvested by centrifugation and lysed using BugBuster™ (Cat #70922, Novagen) in the presence of protease inhibitors. MBP/B. burgdorferi fusion proteins were purified to more than 95% purity using amylose resins (NEB #E8021 L) according to the manufacturer's recommendations (see FIG. 10). To separate B. burgdorferi proteins from MBP; purified MBP fusion proteins were digested with Tobacco Etch Virus (TEV) protease that recognizes a specific consensus site introduced between the MBP and B. burgdorferi proteins during the cloning step. HPLC purification of individual cleaved B. burgdorferi proteins proved to be time-consuming and required extensive optimization of different conditions. To forgo a delay in project implementation, MBP/B. burgdorferi fusion proteins were used in the development of the lateral flow strips inclusive of recombinant MBP protein acting as a negative control. Our data show that none of the human serum samples reacted with MBP, however, one sample of the 40 blind samples showed false positive reactivity to MBP.

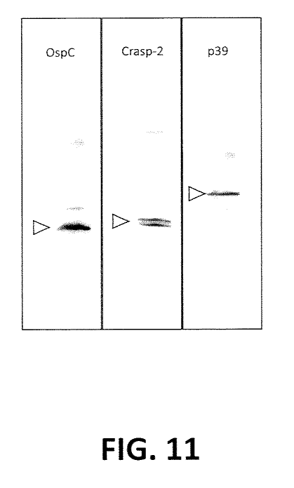

FLAG-fusion proteins were also tested for their ability to eliminate difficulties associated with MBP-fusion proteins including problems with TEV protease cleavage and subsequent purification. FIG. 11 shows Osp C, Crasp-2, and p39 expressed as FLAG-fusion proteins in E. coli. This shows that FLAG-fusion proteins can be used as an alternative expression method to MBP-fusion proteins.

### EXAMPLE 2

### Assay Optimization

Initial investigations to determine assay-specific parameters included the evaluation of human LD positive serum reactivity in a slot blot assay. Recombinant MBP/B. burgdorferi antigens as well as recombinant MBP at known concentrations ranging up to 1 μg were individually loaded into separate slots and adsorbed onto a nitrocellulose membrane overnight at 4° C. The membrane was rotated by 90° prior to application of 1:100 diluted serum from clinically characterized individual donors (n=12), obtained from SeraCare Diagnostics (Milford, Mass.), across all 14 B. burgdorferi antigens (OspA, OspB, OspC, OspE, VlsE, CRASP-1, CRASP-2, DbpA, DbpB, Flagellin, Arp37, P27, P35, and P39). Sera were incubated for 2 h at 4° C. prior to washing of the membrane and application of either HRP-conjugated anti-human IgG Fc or HRP-conjugated anti-human IgM Fc5μ antibodies. Results obtained from individual serum donors after substrate addition showed reactivity to specific B. burgdorferi antigens with no detection of recombinant MBP (data not shown). Negative control sera showed no reactivity to the panel of B. burgdorferi antigens.

Development of the prototype lateral flow assay strip involved the: i) generation of colloidal gold-conjugated MBP/B. burgdorferi antigens; ii) immobilization of fourteen (14) B. burgdorferi antigens on the nitrocellulose membranes at concentrations of 1 μg; and iii) inclusion of recombinant MBP as a negative control on one strip and the inclusion of a flow control band on the other strip. Recombinant MBP/B. burgdorferi antigens were imprinted on the membrane (7 per strip) to reflect their expression during the in vivo life cycle. VlsE, Crasp-1, OspC, p39, OspE, p27, Arp37 and the flow control band constitute test strip 1; whereas Crasp-2, DbpA, OspA, Flagellin, p35, OspB, DbpB and the MBP negative control constitute test strip 2 (FIG. 11).

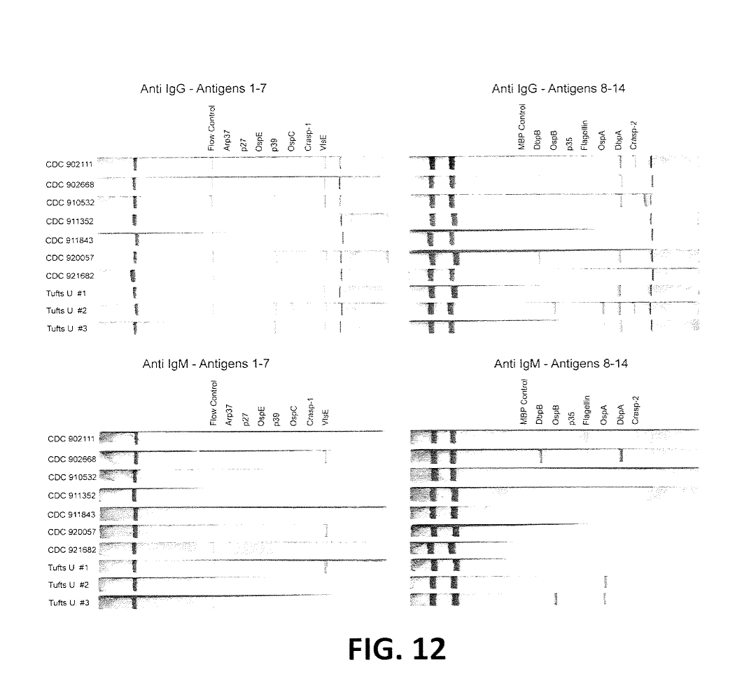

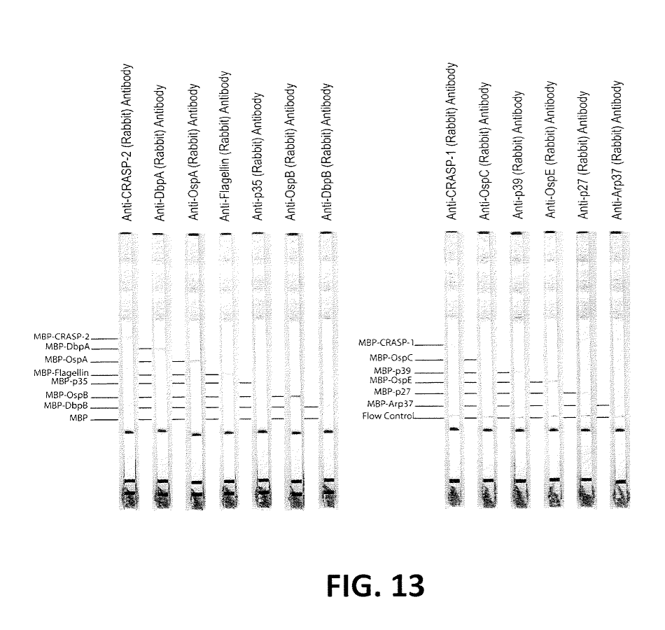

In addition, rabbit polyclonal antibodies were raised against individual B. burgdorferi antigens. All antibodies have been purified using Protein A resin and cross-absorbed against MBP-immobilized resins to eliminate cross-reactivity. These antibodies were used during the optimization and validation of the lateral flow strips as shown in FIG. 12, where immobilized antigens react specifically with the respective rabbit antibody. The data presented in FIG. 8 further demonstrated that (i) B. burgdorferi antigens were properly immobilized on the strip; (ii) gold colloid particles were properly conjugated to respective antigens; (iii) the antibody/gold conjugated antigen complex specifically reacts with respective membrane immobilized antigen and there is no non-specific cross-reactivity and (iv) the lateral flow assay perform correctly.

Preliminary assay performance was determined on lateral flow test strips including the optimization of the conjugate pad system containing impregnated gold-labeled MBP/B. burgdorferi antigens and the imprinted membrane system. The performance capabilities of the assay, including the sensitivity and specificity were evaluated using clinically characterized LD positive human serum samples (n=15) obtained from SeraCare Diagnostics (Milford, Mass.) and negative control sera (data not shown).

### EXAMPLE 3

### Application of the Assay to Measure Borrelia-Specific Antibodies in Human Serum and Comparison with Current Assays Available on the Market

LD positive human sera were obtained from the CDC (n=35) and Tufts University (n=12). Samples from the CDC were accompanied by written results of a clinical exam as well as bioMérieux ELISA and MarBlot IgG and IgM WB results. LD positive human sera and negative control sera (n=5) were analyzed to demonstrate detection of strip immobilized B. burgdorferi antigens by antibodies present in sera of patients diagnosed with LD. IgG and IgM test strips were modified to receive sera premixed with liquid gold-labeled recombinant B. burgdorferi antigens and processed using all 52 sera. Antibody reactivity resulted in gold deposition for band visualization and was recorded after 20 min while wet and after 18 h when dried. No change in band reactivity between the two evaluation time points was noted. All LD positive sera showed significant reactivity to several B. burgdorferi antigens, with some samples reacting with 10-12 of the 14 immobilized antigens. Strips reactive with sera from patients diagnosed with early/acute LD showed a greater number of reactive bands in IgM strips relative to IgG strips, as shown in FIG. 11. Conversely, sera from patients diagnosed with chronic or late stage LD showed a greater number of reactive bands in IgG strips relative to IgM strips. This reactivity pattern is consistent with the ability of the assay to differentiate between stages of the disease which manifest different antibody populations within LD positive patient sera. Of particular note are three (3) LD positive sera obtained from the CDC which are positive by clinical exam but negative by ELISA and MarBlot IgG and IgM tests. These three samples are positive by the present invention. This indicates that the present invention shows significantly less false negative results than tests commonly in use today.

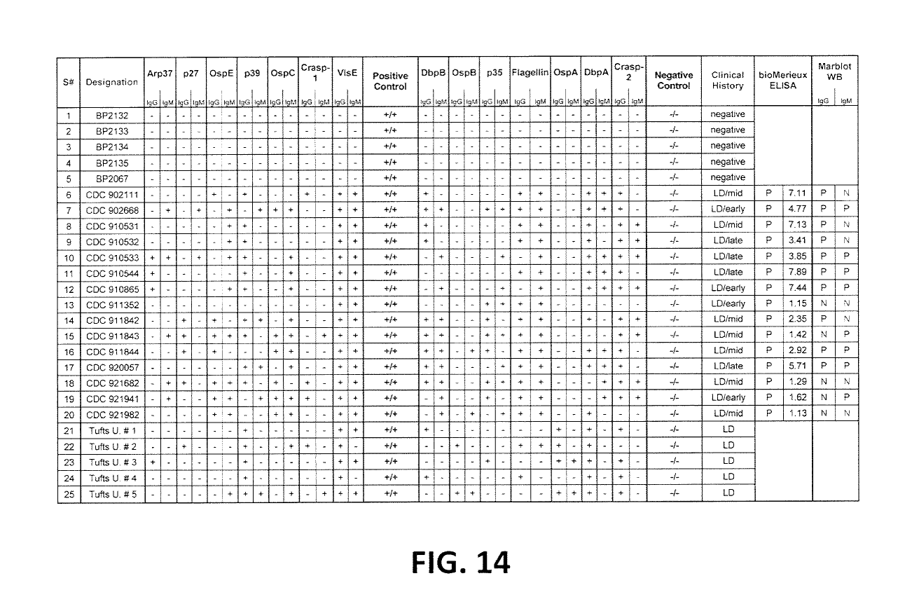

Positive LD sera showed 100% reactivity to LD antigens as indicated by detection of between 4 to 8 antigens per strip and a 0% false negative rate. By comparison, a 27% false negative rate is observed for the MarBlot IgG test from patients diagnosed with mid and late stage LD and a 25% false negative rate is observed for the MarBlot IgM test from patients diagnosed with early stage LD. Furthermore, 60% of the LD sera provided by the CDC showed weak reactivity in the bioMérieux ELISA as defined by a score of <4.0; nonetheless, all of these sera showed more than 4 reactive antigens in the IgG or IgM test strips of the present invention. This finding indicates that the present invention is potentially a more robust assay than current commercially available assays for the detection of LD reactive antibodies present in the sera of patients diagnosed with the disease. Data is summarized in FIG. 14.

The data confirms earlier findings, and most importantly, accurately identifies LD negative samples where the current commercial C₆ VslE test registers false positive results. We analyzed additional sera using 40 coded ‘blind’ samples. As before, samples applied to the present invention were subsequently scored for positive or negative reactivity to LD antigens. Once scored, the results of the present invention were compared to the results for each sample for the C₆ VlsE ELISA test (Table 1). In instances where results of the two tests differed, either the present invention showed a high false negative rate or the C₆ VlsE ELISA test showed a high false positive rate. To determine which test showed greater specificity, the results of WB testing and clinical exams (if available) were released for comparison (Table 2).

TABLE 1
Data obtained from the evaluation of 40 “blind” samples provided. After
scoring, the results of the present invention were compared to C₆ VlsE
ELISA results. C₆ VlsE ELISA results indicated as POSITIVE* may
represent either a high false negative rate for the present invention or a
high false positive rate for the C₆ VlsE ELISA test. One of the test of the
present invention scored POSITIVE^{†} was determined to be a
false positive result due to MBP reactivity with patient sera.

| | Present | Present | | |
|---|---|---|---|---|
| | Invention | Invention | | C₆ VlsE |
| Blind | Test IgG | Test IgM | Present Invention | ELISA |
| Sample # | Score | Score | Test Assessment | Results |
| 1 | positive | positive | Early/mid stage LD | positive |
| 2 | negative | negative | LD negative | negative |
| 5 | negative | negative | LD negative | negative |
| 6 | negative | negative | LD negative | negative |
| 7 | positive | negative | Mid/late stage LD | positive |
| 9 | positive | negative | Mid/late stage LD | positive |
| 10 | negative | negative | LD negative | negative |
| 11 | negative | negative | LD negative | negative |
| 12 | negative | negative | LD negative | negative |
| 18 | positive | positive | Mid/late stage LD | positive |
| 22 | negative | negative | LD negative | POSITIVE* |
| 23 | negative | negative | LD negative | POSITIVE* |
| 24 | negative | negative | LD negative | POSITIVE* |
| 25 | negative | negative | LD negative | negative |
| 26 | negative | POSITIVE^{†} | LD negative | negative |
| 30 | positive | negative | Mid/late stage LD | positive |
| 31 | positive | negative | Mid/late stage LD | positive |
| 36 | negative | negative | LD negative | negative |
| 37 | negative | negative | LD negative | negative |
| 39 | positive | negative | Mid/late stage LD | positive |
| 42 | negative | negative | LD negative | POSITIVE* |
| 43 | positive | negative | Mid/late stage LD | positive |
| 44 | positive | negative | Mid/late stage LD | positive |
| 45 | negative | negative | LD negative | negative |
| 49 | positive | positive | Early/mid stage LD | positive |
| 55 | negative | negative | LD negative | negative |
| 57 | positive | negative | Mid/late stage LD | positive |
| 58 | negative | negative | LD negative | negative |
| 59 | negative | negative | LD negative | negative |
| 60 | negative | negative | LD negative | POSITIVE* |
| 61 | negative | negative | LD negative | POSITIVE* |
| 65 | negative | negative | LD negative | negative |
| 67 | negative | negative | LD negative | POSITIVE* |
| 68 | negative | negative | LD negative | negative |
| 69 | negative | negative | LD negative | negative |
| 70 | negative | negative | LD negative | negative |
| 71 | negative | negative | LD negative | POSITIVE* |
| 72 | negative | negative | LD negative | POSITIVE* |
| 74 | negative | negative | LD negative | negative |
| 75 | negative | negative | LD negative | negative |

TABLE 2
C₆ VlsE ELISA results indicated as POSITIVE* in Table 2 were deemed to be false positive
results when WB and clinical exam findings (when available) were used for confirmation.
The present invention correctly identified these 9 samples as LD negative samples
in contrast to the current commercial C6 VslE test which registered false positive results.

| | Present | Present | Present | | Western | |
|---|---|---|---|---|---|---|
| Blind | Invention | Invention | Invention | C₆ VlsE ELISA | Blot | Clinical |
| Sample # | IgG Score | IgM Score | Assessment | Results | Results | Findings |
| 22 | negative | negative | LD negative | FALSE POSITIVE | negative | |
| 23 | negative | negative | LD negative | FALSE POSITIVE | negative | |
| 24 | negative | negative | LD negative | FALSE POSITIVE | negative | |
| 42 | negative | negative | LD negative | FALSE POSITIVE | negative | |
| 60 | negative | negative | LD negative | FALSE POSITIVE | negative | negative |
| 61 | negative | negative | LD negative | FALSE POSITIVE | negative | |
| 67 | negative | negative | LD negative | FALSE POSITIVE | negative | |
| 71 | negative | negative | LD negative | FALSE POSITIVE | negative | negative |
| 72 | negative | negative | LD negative | FALSE POSITIVE | negative | |

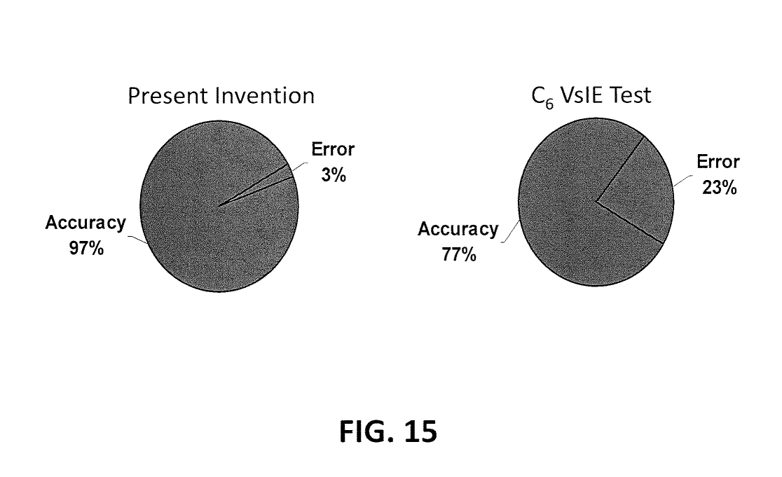

We conclude that the present invention shows 97.5% accuracy with a 2.5% false positive rate due to 1 sample showing false positive results caused by MBP cross reactivity and a 0% false negative rate. Furthermore, in comparison the C₆ VslE test showed 9 samples with false positive results (77.5% accuracy) which the present invention correctly identified as LD negative when compared to WB results and clinical findings. The present invention also differentiated between early/acute and late/chronic stage LD based on IgG vs. IgM reactivity and differential detection of LD antigens present in our test (data not shown). This data, collected using “blind” samples, includes additional negative controls and confirms the results we collected previously. This demonstrates the greater specificity of the present invention compared to the ELISA test currently in use today. Accuracy of both the present invention and C₆ VslE test for these samples is summarized in FIG. 15.

Although the invention has been described in detail for the purpose of illustration, it is to be understood that such detail is solely for that purpose and that variations can be made therein by those skilled in the art without departing from the spirit and scope of the invention.

What is claimed is:

1. A composition for the detection of B. burgdorferi or Lyme disease comprising at least two proteins selected from the group consisting of OspA (SEQ ID NO: 1), OspB (SEQ ID NO: 2), OspC (SEQ ID NO: 3), OspE (SEQ ID NO: 4), VlsE (SEQ ID NO: 5), CRASP-1 (SEQ ID NO: 6), CRASP-2 (SEQ ID NO: 7), DbpA (SEQ ID NO: 8), DbpB (SEQ ID NO: 9), Flagellin (SEQ ID NO: 10), Arp37 (SEQ ID NO: 11), P27 (SEQ ID NO: 12), P35 (SEQ ID NO: 13), P39 (SEQ ID NO: 14), P66 (SEQ ID NO: 15), BBK32 (SEQ ID NO: 16), and RevA (SEQ ID NO: 17).

2. The composition of claim 1, wherein the at least two proteins are conjugated to a label.

3. The composition of claim 2, wherein the label is gold, peroxidase, biotin, or a fluorescent dye.

4. The composition of claim 1, wherein one of the at least two proteins are selected from the group consisting of OspA, OspB, OspC, OspE, p27, p66, BBK32, and RevA.

5. The composition of claim 1, wherein one of the at least two proteins are selected from the group consisting of OspE, Flagellin, VlsE, CRASP-1, CRASP-2, p35, p27 and BBK32.

6. The composition of claim 1, wherein one of the at least two proteins are selected from the group consisting of Flagellin, VlsE, CRASP-1, CRASP-2, p35, p27, BBK32, DbpA, DbpB, Arp37, p39 and p66.

7. A test strip for the detection of B. burgdorferi comprising a solid support having thereon
a. a sample pad;
b. a label pad adjacent to the sample pad;
c. a membrane adjacent to the label pad, said membrane contains at least two proteins immobilized thereon, wherein the at least two proteins are selected from the group consisting of OspA (SEQ ID NO: 1), OspB (SEQ ID NO: 2), OspC (SEQ ID NO: 3), OspE (SEQ ID NO: 4), VlsE (SEQ ID NO: 5), CRASP-1 (SEQ ID NO: 6), CRASP-2 (SEQ ID NO: 7), DbpA (SEQ ID NO: 8), DbpB (SEQ ID NO: 9), Flagellin (SEQ ID NO: 10), Arp37 (SEQ ID NO: 11), P27 (SEQ ID NO: 12), P35 (SEQ ID NO: 13), P39 (SEQ ID NO: 14), P66 (SEQ ID NO: 15), BBK32 (SEQ ID NO: 16), and RevA (SEQ ID NO: 17); and
d. a wick adjacent to the membrane.

8. The test strip of claim 7, wherein the label pad comprises label-conjugated proteins, wherein the proteins are the same as the at least two proteins.

9. The test strip of claim 8, wherein the label is gold, peroxidase, biotin, or a fluorescent dye.

10. The test strip of claim 7, wherein the at least two proteins immobilized on the membrane are physically separated from each other.

11. The test strip of claim 7, further comprising a flow control antibody.

12. The test strip of claim 7, wherein one of the at least two proteins are a) selected from the group consisting of OspA, OspB, OspC, OspE, p27, p66, BBK32, and RevA; b) selected from the group consisting of OspE, Flagellin, VlsE, CRASP-1, CRASP-2, p35, p27 and BBK32; or c) selected from the group consisting of Flagellin, VlsE, CRASP-1, CRASP-2, p35, p27, BBK32, DbpA, DbpB, Arp37, p39 and p66.

13. A method for detecting B. burgdorferi or diagnosing Lyme disease comprising the steps of
a. providing a biological fluid sample from an individual;
b. contacting the fluid sample with at least two proteins selected from the group consisting of OspA (SEQ ID NO: 1), OspB (SEQ ID NO: 2), OspC (SEQ ID NO: 3), OspE (SEQ ID NO: 4), VlsE (SEQ ID NO: 5), CRASP-1 (SEQ ID NO: 6), CRASP-2 (SEQ ID NO: 7), DbpA (SEQ ID NO: 8), DbpB (SEQ ID NO: 9), Flagellin (SEQ ID NO: 10), Arp37 (SEQ ID NO: 11), P27 (SEQ ID NO: 12), P35 (SEQ ID NO: 13), P39 (SEQ ID NO: 14), P66 (SEQ ID NO: 15), BBK32 (SEQ ID NO: 16), and RevA (SEQ ID NO: 17);
c. detecting antibody-antigen complexes containing the at least two proteins, wherein the presence of the antibody-antigen complexes indicate the presence of B. burgdorferi or Lyme disease.

14. The method of claim 13, wherein antibodies in the sample is labeled before the contacting step.

15. The method of claim 13, wherein the fluid sample is a blood sample.

16. The method of claim 13, wherein the method is carried out using ELISA or a test strip.

17. The method of claim 13, further comprising the step of
a. diagnosing early stage Lyme when antibody-antigen complexes are detected containing at least two proteins selected from the group consisting of OspA (SEQ ID NO: 1), OspB (SEQ ID NO: 2), OspC (SEQ ID NO: 3), OspE (SEQ ID NO: 4), P27 (SEQ ID NO: 12), P66 (SEQ ID NO: 15), BBK32 (SEQ ID NO: 16), and RevA (SEQ ID NO: 17);
b. diagnosing middle stage Lyme when antibody-antigen complexes are detected containing at least two proteins selected from the group consisting of OspE (SEQ ID NO: 4), Flagellin (SEQ ID NO: 10), VlsE (SEQ ID NO: 5), CRASP-1 (SEQ ID NO: 6), CRASP-2 (SEQ ID NO: 7), P35 (SEQ ID NO: 13), P27 ((SEQ ID NO: 12), and BBK32 (SEQ ID NO: 16); or
c. diagnosing late stage Lyme when antibody-antigen complexes are detected containing at least two proteins selected from the group consisting of Flagellin (SEQ ID NO: 10), VlsE (SEQ ID NO: 5), CRASP-1 (SEQ ID NO: 6), CRASP-2 (SEQ ID NO: 7), P35 (SEQ ID NO: 13), P27 (SEQ ID NO: 12), BBK32 (SEQ ID NO: 16), DbpA (SEQ ID NO: 8), DbpB (SEQ ID NO: 9), Arp37 (SEQ ID NO: 11), P39 (SEQ ID NO: 14), and P66 (SEQ ID NO: 15).

18. A kit for the detection of detection of B. burgdorferi or Lyme disease comprising
a. at least two proteins selected from the group consisting of OspA (SEQ ID NO: 1), OspB (SEQ ID NO: 2), OspC (SEQ ID NO: 3), OspE (SEQ ID NO: 4), VlsE (SEQ ID NO: 5), CRASP-1 (SEQ ID NO: 6), CRASP-2 (SEQ ID NO: 7), DbpA (SEQ ID NO: 8), DbpB (SEQ ID NO: 9), Flagellin (SEQ ID NO: 10), Arp37 (SEQ ID NO: 11), P27 (SEQ ID NO: 12), P35 (SEQ ID NO: 13), P39 (SEQ ID NO: 14), P66 (SEQ ID NO: 15), BBK32 (SEQ ID NO: 16), and RevA (SEQ ID NO: 17); and
b. antibodies specific for the at least two proteins.

19. The kit of claim 18, wherein the at least two proteins are conjugated to a label.

20. The kit of claim 18, wherein the at least four proteins are immobilized on a membrane.


SEQUENCE LISTING (17 sequences)

SEQ ID NO: 1 - protein, 273 aa, Borrelia burgdorferi
MKKYLLGIGLILALIACKQNVSSLDEKNSVSVDLPGEMNVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTEIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 2 - protein, 296 aa, Borrelia burgdorferi
MRLLIGFALALALIGCAQKGAESIGSQKENDLNLEDSSKKSHQNAKQDLPAVTEDSVSLFNGNKIFVSKEKNSSGKYDLRATIDQVELKGTSDKNNGSGTLEGSKPDKSKVKLTVSADLNTVTLEAFDASNQKISSKVTKKQGSITEETLKANKLDSKKLTRSNGTTLEYSQITDADNATKAVETLKNSIKLEGSLVVGKTTVEIKEGTVTLKREIEKDGKVKVFLNDTAGSNKKTGKWEDSTSTLTISADSKKTKDLVFLTDGTITVQQYNTAGTSLEGSASEIKNLSELKNALK

SEQ ID NO: 3 - protein, 210 aa, Borrelia burgdorferi
MKKNTLSAILMTLFLFISCNNSGKDGNTSANSADESVKGPNLTEISKKITDSNAVLLAVKEVEALLSSIDEIAAKAIGKKIHQNNGLDTENNHNGSLLAGAYAISTLIKQKLDGLKNEGLKEKIDAAKKCSETFTNKLKEKHTDLGKEGVTDADAKEAILKTNGTKTKGAEELGKLFESVEVLSKAAKEMLANSVKELTSPVVAESPKKP

SEQ ID NO: 4 - protein, 177 aa, Borrelia burgdorferi
MEKFMNKKMKMFIICAVFILIGACKIHTSYDEQSNGEVKVKKIEFSEFTVKIKNKNNSNNWADLGDLVVRKEKDGIETGLNAGGHSATFFSLEEEEINNFIKAMTEGGSFKTSLYYGYNDEESDKNVIKNKEIKTKIEKINDTEYITFLGDKINNSAGGDKIAEYAISLEELKRNLK

SEQ ID NO: 5 - protein, 357 aa, Borrelia burgdorferi
MNTKKISSAILLTTFFVFINCKSQVADKDDPTNKFYQSVIQLGNGFLDVFTSFGGLVAEAFGFKSDPKKSDVKTYFTTVAAKLEKTKTDLNSLPKEKSDISSTTGKPDSTGSVGTAVEGAIKEVSELLDKLVKAVKTAEGASSGTAAIGEVVDNAAAAKAADKDSVTGIAKGIKEIVEAAGGSKKLKAAAAKGENNKGAGKLFGKAGDAAHGDSEAASKAAGAVSAVSGEQILSAIVKAAAAGDQEGKKPGEAKNPIAAAIGEGDGDAEFNQDGMKKDDQIAAAIALRGMAKDGKFAVKNDEKGKAEGAIKGAAESAVRKVLGAITGLIGDAVSSGLRKVGDSVKAASKETPPALNK

SEQ ID NO: 6 - protein, 251 aa, Borrelia burgdorferi
LKKAKLNIIKINIIAMILTLICTSCAPFSKIDPKANANTKPKKITNPGENTQNFEDKSGDLSTSDEKIMETIASELKAIGKELEDQKKEENIQIAKIAKEKFDFLSTFKVGPYDLIDEDIQMKIKRTLYSSLDYKKENIEKLKEILEILKKNSEHYNIIGRLIYHISWGIQFQIEQNLELIQNGVENLSQEESKSLLMQIKSNLEIKQRLKKTLNETLKVYNQNTQDNEKILAEHFNKYYKDFDTLKPAFY

SEQ ID NO: 7 - protein, 236 aa, Borrelia burgdorferi
MKKSFLSIYMLISISLLSCDVSRLNQRNINELKIFVEKAKYYSIKLDAIYNECTGAYNDIMTYSEGTFSDQSKVNQAISIFKKDNKIVNKFKELEKIIEEYKPMFLSKLIDDFAIELDQAVDNDVSNARHVADSYKKLRKSVVLAYIESFDVISSKFVDSKFVEASKKFVNKAKEFVEENDLIALECIVKTIGDMVNDREINSRSRYNNFYKKEADFLGAAVELEGAYKAIKQTLL

SEQ ID NO: 8 - protein, 191 aa, Borrelia burgdorferi
MIKCNNKTFNNLLKLTILVNLLISCGLTGATKIRLERSAKDITDEIDAIKKDAALKGVNFDAFKDKKTGSGVSENPFILEAKVRATTVAEKFVIAIEEEATKLKETGSSGEFSAMYDLMFEVSKPLQKLGIQEMTKTVSDAAEENPPTTAQGVLEIAKKMREKLQRVHTKNYCTLKKKENSTFTDEKCKNN

SEQ ID NO: 9 - protein, 187 aa, Borrelia burgdorferi
MKIGKLNSIVMVLFFDLLVACSIGLVERTNAALESSSKDLKNKILKIKKEATGKGVLFEAFTGLKTGSKVTSGGLALREAKVQAIVETGKFLKIIEEEALKLKETGNSGQFLAMFDLMLEVVESLEDVGIIGLKARVLEESKNNPINTAERLLAAKAQIENQLKVVKEKQNIENGGEKKNNKSKKKK

SEQ ID NO: 10 - protein, 336 aa, Borrelia burgdorferi
MIINHNTSAINASRNNGINAANLSKTQEKLSSGYRINRASDDAAGMGVSGKINAQIRGLSQASRNTSKAINFIQTTEGNLNEVEKVLVRMKELAVQSGNGTYSDADRGSIQIEIEQLTDEINRIADQAQYNQMHMLSNKSASQNVRTAEELGMQPAKINTPASLSGSQASWTLRVHVGANQDEAIAVNIYAANVANLFSGEGAQTAQAAPVQEGVQQEGAQQPAPATAPSQGGVNSPVNVTTTVDANTSLAKIENAIRMISDQRANLGAFQNRLESIKNSTEYAIENLKASYAQIKDATMTDEVVAATTNSILTQSAMAMIAQANQVPQYVLSLLR

SEQ ID NO: 11 - protein, 332 aa, Borrelia burgdorferi
MKLLKIFMCAFLLLNLVNCKFDSLNLSTKSVDDKNNSIAKLLQHLSKSEDQANKTSTSEDQKELEITENKEQEHEKLSQVAQHAPNSKIEKVKSDGKPVPGDKILSSNKDIYNSYIPEVKEEIVYEILEEVIIPETKIPEITEEVIMPIPQTIDFYIEPRPISSFLTQGTSPSITSTIKSYKELAKEKINNGLNIVQKITQNIDNITENLNSKETPKEISGKEVEEKITHPIFDHITGSGNNPGQDSISNTWGEGLEIGGDSNFFTNLEEVRSSIRTKIKVSDGTEQTKDKVEIDEIIEDLQKLKEFLEKLKKYLKDTNNLSAIEESVKGLS

SEQ ID NO: 12 - protein, 277 aa, Borrelia burgdorferi
MSKKVILILLEILILSCDLSINKEQKTKEKTSEKQESEKQNIEKQEPEKQKQNAAKIIPTVSIQTVEIRESNQIPKSIEKYYKQAYPIQTFTLDFSITREKEFLKPEDKILPTQGKVESLSILINKKLLDFKAPENPKSSTLKNFKEIKNIENFFQNQDLLFVLTLKDKNNNNTINIMLNPPNDIQKPKDYILKDLKDTIKKGTGEKYLNPIYRFQIKNKKDYHSIDYNKVTISEKTIELDLLPHEQVFQMNKNFTKILDTITDLNNLKLVIQKELV

SEQ ID NO: 13 - protein, 249 aa, Borrelia burgdorferi
MKYNTIISIFVCLFLTACNPDFNTNKKRTLSKGIISNQDADSDKIIKNKLLDDLINLIEKANADREKYVKKMEEEPSDQYGMLAVFGGMYWAESPRELISDTGSERSIRYRRRVYSILLNAIETNELKKFSEIRILSIKVLEIFSLFNLFGSTLDDVVVHLYSKKDTLGKLDISNLKRLKNLFEKLLSIKTIVSKMSKRLLLDYQNNENFIKTDNAKLGSYVVALSNQIQEKYNEAERLKSEIILIYTL

SEQ ID NO: 14 - protein, 339 aa, Borrelia burgdorferi
MNKILLLILLESIVFLSCSGKGSLGSEIPKVSLIIDGTFDDKSFNESALNGVKKVKEEFKIELVLKESSSNSYLSDLEGLKDAGSDLIWLIGYRFSDVAKVAALQNPDMKYAIIDPIYSNDPIPANLVGMTFRAQEGAFLTGYIAAKLSKTGKIGFLGGIEGEIVDAFRYGYEAGAKYANKDIKISTQYIGSFADLEAGRSVATRMYSDEIDIIHHAAGLGGIGAIEVAKELGSGHYIIGVDEDQAYLAPDNVITSTTKDVGRALNIFTSNHLKTNTFEGGKLINYGLKEGVVGFVRNPKMISFELEKEIDNLSSKIINKEIIVPSNKESYEKFLKEFI

SEQ ID NO: 15 - protein, 618 aa, Borrelia burgdorferi
MKSHILYKLIIFLTTSAAIFAADALKEKDIFKINPWMPTFGFENTSEFRLDMDELVPGFENKSKITIKLKPFEANPELGKDDPFSAYIKVEDLALKAEGKKGDQFKIDVGDITAQINMYDFFIKISTMTDFDFNKESLFSFAPMTGFKSTYYGFPSNDRAVRGTILARGTSKNIGTIQLGYKLPKLDLTFAIGGTGTGNRNQENDKDTPYNKTYQGILYGIQATWKPIKNLLDQNEDTKSVIAETPFELNFGLSGAYGNETFNNSSITYSLKDKSVVGNDLLSPTLSNSAILASFGAKYKLGLTKINDKNTYLILQMGTDFGIDPFASDFSIFGHISKAANFKKETPSDPNKKAEIFDPNGNALNFSKNTELGIAFSTGASIGFAWNKDTGEKESWAIKGSDSYSTRLFGEQDKKSGVALGISYGQNLYRSKDTEKRLKTISENAFQSLNVEISSYEDNKKGIINGLGWITSIGLYDILRQKSVENYPTTISSTTENNQTEQSSTSTKTTTPNLTFEDAMKLGLALYLDYAIPIASISTEAYVVPYIGAYILGPSNKLSSDATKIYLKTGLSLEKLIRFTTISLGWDSNNIIELANKNTNNAAIGSAFLQFKIAYSGS

SEQ ID NO: 16 - protein, 360 aa, Borrelia burgdorferi
MKKVKSKYLALGLLFGFISCDLFIRYEMKEESPGLFDKGNSILETSEESIKKPMNKKGKGKIARKKGKSKVSRKEPYIHSLKRDSANKSNFLQKNVILEEESLKTELLKEQSETRKEKIQKQQDEYKGMTQGSLNSLSGESGELKETIESNEIDITIDSDLRPKSSLQDIAGSNSISYTDEIEEEDYARYYLDEDDEDDEYYEDDYEEIRLSNRYQSYLEGVKYNVDSAINTINKIYDTYTLFSTKLTQMYSTRLDNLAKAKAKEEAAKFTKEDLEKNFKTLLNYIQVSVKTAANFVYINDTHAKRKLENIEAEIKTLIAKIKEQSNLYEAYKAIVTSILLMRDSLKEVQGIIDKNGVWY

SEQ ID NO: 17 - protein, 160 aa, Borrelia burgdorferi
MRNKNIFKLFFASMLFVMACKAYVEEKKEIDSLMEDVLALVNDSSGGKFKDYKDKINELKENLKDIGNAELKEKLLNLQNSFQDKLAAKLAALKAAKNTIENITDKDQDISKRKIWSEAKLVGVTVPLLGSNTSGNGDKMSKNAVEQIDKVIKFLEEGTN